(19) United States
(12) Patent Application Publication
(10) Pub. No.: US 2022/0016290 A1
(43) Pub. Date: Jan. 20, 2022

(54) SYSTEM AND METHOD FOR REGULATING THE TEMPERATURE OF THE CABIN OF AN AIRCRAFT WHEN ON THE GROUND

(71) Applicants: Airbus SAS, BLAGNAC (FR); Airbus Defence and Space GmbH, TAUFKIRCHEN (DE); Airbus Operations GmbH, HAMBURG (DE)

(72) Inventors: Eloi CHABAUD, BLAGNAC (FR); Philippe CHANTAL, BLAGNAC (FR); Svetoslav DUHOVNIKOV, TAUFKIRCHEN (DE); Dariusz KRAKOWSKI, HAMBURG (DE); Martin KUBISCH, TAUFKIRCHEN (DE)

(21) Appl. No.: 17/379,525

(22) Filed: Jul. 19, 2021

(30) Foreign Application Priority Data
Jul. 20, 2020 (FR) 2007566

(51) Int. Cl. A61L 2/24; A61L 9/015; B64D 13/08; B64F 5/30; G06K 7/14

(52) U.S. Cl. CPC A61L 2/24; A61L 9/015; B64D 13/08; B64F 5/30; B64D 2013/064; A61L 2202/14; A61L 2202/25; A61L 2209/111; G06K 7/1417

(57) ABSTRACT

The system includes a computer server receiving data representative of the temperature of the cabin and including a database containing, for the aircraft, a predetermined cycle of regulation of the temperature of the air of the cabin; a ground pre-conditioned air unit including: a pre-conditioned air generator and a control module; and a user interface connected to the computer server and transmitting a control signal to the control module; and the control module, upon receiving the control signal, controlling the pre-conditioned air generator, according to the predetermined cycle, which is modulated depending on the data. This system makes it possible to control the temperature of the cabin in real time, so as to correctly disinfect it.

### CROSS-REFERENCES TO RELATED APPLICATIONS

This application claims the benefit of the French patent application No. 2007566 filed on Jul. 20, 2020, the entire disclosures of which are incorporated herein by way of reference.

### FIELD OF THE INVENTION

The present application relates to a system and a method for regulating the temperature of the cabin of an aircraft when on the ground, permitting effective disinfection of the whole cabin.

### BACKGROUND OF THE INVENTION

Nowadays, in spite of certain sanitary precautions, passengers of an aircraft can be infected with a virus that is transmissible, in particular via contact surfaces in the cabin. Thus, a cabin is at risk of being exposed to infected passengers.

In order to avoid the spread of viruses, cabins are disinfected on a regular basis.

It is known that certain viruses cease to be active if exposed to a predetermined temperature for a given duration.

In order to bring a cabin of an aircraft to the predetermined temperature, and thus to disinfect the cabin, use is generally made of a ground pre-conditioned air (PCA) unit.

FIG. 1 shows an aircraft 10 on the ground, to which a pre-conditioned air unit 12, external to the aircraft 10, is connected. The pre-conditioned air unit 12 comprises a pre-conditioned air generator 14 which is fluidically connected, via a flexible air line 16, to the air ducts (not shown in FIG. 1) of the aircraft 10. The pre-conditioned air generator 14 is configured to distribute air into the cabin according to a predetermined cycle for regulating the temperature of the air of the cabin for a given duration. The pre-conditioned air unit 12 can be controlled manually by a user, who activates the pre-conditioned air generator 14 and initiates the predetermined cycle. A regulating cycle is defined by a first phase of raising (or lowering) the temperature from an initial temperature, until a predetermined temperature is reached, then a second phase of holding at the predetermined temperature for a given duration, and finally a third phase of lowering (or raising) from the predetermined temperature to a final temperature which corresponds to the initial temperature.

However, when using a pre-conditioned air unit 12 of that kind, the temperature of the cabin is not homogeneous throughout the cabin. Indeed, certain zones of the cabin of the aircraft may, for example, not reach the predetermined temperature during the entire given duration. This means that, after a disinfection cycle, some zones of the cabin may still contain an active virus.

The present invention aims to propose a solution by which it is possible to optimize the disinfection of a cabin of an aircraft.

### SUMMARY OF THE INVENTION

To that end, the invention relates to a system for regulating the temperature of the cabin of an aircraft when on the ground.

According to the invention, the system comprises:

a plurality of sensors arranged in various zones of the cabin, each sensor being configured to acquire, in real time, data representative of the temperature of the zone of the cabin,

a computer server configured to receive, in real time, the data from the plurality of sensors and comprising a database containing, for the aircraft, a predetermined cycle of regulation of the temperature of the air of the cabin to a predetermined temperature during a given duration,

a ground pre-conditioned air unit comprising:

a pre-conditioned air generator for generating pre-conditioned air in the cabin, and

a control module connected to the pre-conditioned air generator and configured to receive the data from the computer server,

a user interface connected to the computer server, and configured to access the data from the computer server and to transmit a control signal to the control module.

According to the invention, the control module is configured such that, upon receiving the control signal, it controls, in real time, the pre-conditioned air generator, according to the predetermined cycle, the predetermined cycle being modulated, in real time, depending on the data from the computer server.

Advantageously, the system according to the invention makes it possible to control the temperature of the cabin of an aircraft in real time and during a predetermined cycle of regulation of the temperature of the air of the cabin. The system thus makes it possible to verify, in real time, that for each zone of the cabin, the temperature of the cabin reaches a predetermined temperature for a given duration. Since the predetermined cycle corresponds to a cabin disinfection cycle, the system makes it possible to check that all of the zones of the cabin of the aircraft are correctly subjected to the predetermined temperature for the predetermined duration, and thus that all of the zones of the cabin of the aircraft are correctly disinfected.

According to a first embodiment, the user interface is a portable telephone, a tablet or a computer.

According to a second embodiment, the pre-conditioned air unit includes the user interface.

According to one feature, the plurality of sensors is configured to transmit the data to the computer server by wireless transmission. According to this feature, the computer server is configured to transmit the data to the control module by wireless transmission. According to this feature, the user interface is connected to the computer server via a wireless connection.

According to another feature, the control module is configured such that, upon receiving the control signal, it sends data relating to its location to the computer server.

According to another feature, the pre-conditioned air unit comprises a reservoir containing at least one disinfectant solution connected to the pre-conditioned air generator, and the pre-conditioned air generator is configured to generate a mixture comprising pre-conditioned air and the disinfectant solution.

According to another feature, the control module comprises safety means comprising a comparison submodule that is configured to compare the value of each data item from the computer server with a predetermined threshold, and a stopping submodule that is configured to stop the control of the pre-conditioned air generator when the value of at least one data item from the computer server is above the predetermined threshold.

According to another feature, the aircraft is fitted with an identifier. According to this feature, the user interface comprises means for detecting an identifier, and is configured such that, upon detecting the identifier of the aircraft, it accesses the data of the computer server for the identified aircraft.

The invention also relates to a method for regulating the temperature of the cabin of an aircraft when on the ground, using a regulation system comprising a plurality of sensors arranged in various zones of the cabin, a computer server comprising a database containing, for the aircraft, a predetermined cycle of regulation of the temperature of the air of the cabin to a predetermined temperature during a given duration, a user interface connected to the computer server, and a ground pre-conditioned air unit, which comprises a pre-conditioned air generator and a control module connected to the pre-conditioned air generator.

According to the invention, the method comprises the following steps:

acquiring, in real time and by means of the plurality of sensors, data representative of the temperature of the various zones of the cabin,

receiving, in real time, the data from the plurality of sensors by means of the computer server,

accessing, via the user interface, the data from the computer server,

transmitting, via the user interface, a control signal, to the control module, and

upon receiving the control signal:

receiving, by the control module and in real time, the data from the computer server,

modulating, in real time, the predetermined cycle depending on the data from the computer server,

controlling, in real time, the pre-conditioned air generator, according to the modulated predetermined cycle.

According to one feature, the aircraft is fitted with an identifier, and the user interface comprises means for detecting an identifier. According to this feature, the method comprises, prior to the step of accessing the data of the computer server, the steps of:

using the detection means to identify the aircraft by means of its identifier,

transmitting, via the user interface, the identifier of the aircraft to the control module.

The invention also relates to a computer program product, comprising a set of program code instructions that, when the instructions are executed by a processor, configure the processor to implement a method for regulating the temperature of the cabin of an aircraft when on the ground according to the invention.

### BRIEF DESCRIPTION OF THE DRAWINGS

Other features and advantages will emerge from the following description of the invention, which description is given solely by way of example, with reference to the appended drawings in which:

FIG. 1 is a side view of an aircraft to which a pre-conditioned air unit is connected, and illustrates an embodiment of the prior art,

FIG. 2 is a perspective view of a system for regulating the temperature of the cabin of an aircraft that is on the ground, illustrating an embodiment of the invention,

FIG. 3 is a graph illustrating the change over time of the temperature in an aircraft cabin, a regulation system which illustrates an embodiment of the invention being connected to the aircraft,

FIG. 4 is a view of a user interface of a system for regulating the temperature of the cabin of an aircraft that is on the ground, illustrating an embodiment of the invention,

FIG. 5 is a perspective view of a system for regulating the temperature of the cabin of an aircraft that is on the ground, illustrating another embodiment of the invention, and

FIG. 6 is a perspective view of a system for regulating the temperature of the cabin of a fleet of aircraft, illustrating an embodiment of the invention.

### DETAILED DESCRIPTION OF THE PREFERRED EMBODIMENTS

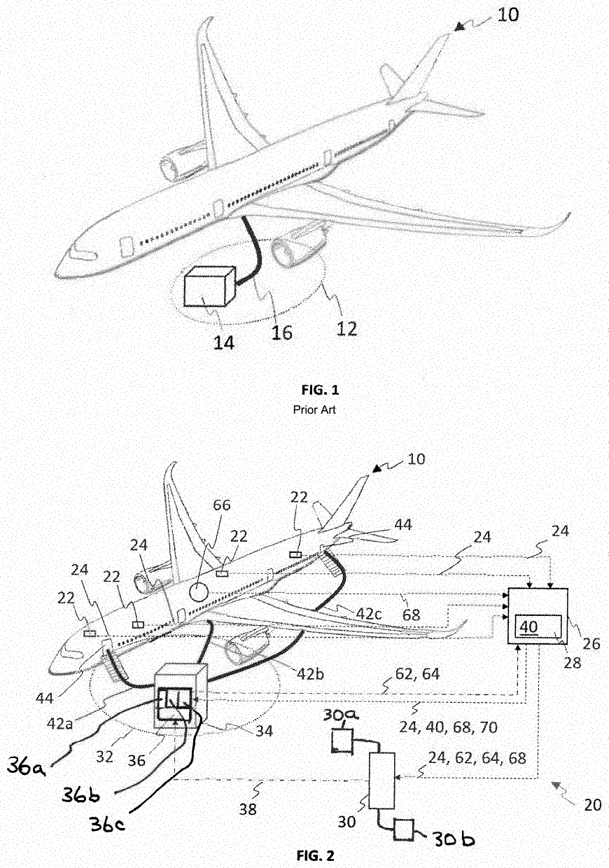

FIG. 2 shows a system 20 for regulating the temperature of the cabin of an aircraft 10.

The “cabin of the aircraft” is to be understood as the interior of the aircraft, that is to say, all of the following: the cockpit of the aircraft, the avionics bays and the cabin containing the passenger seats.

The system 20 comprises a plurality of sensors 22 arranged in the cabin of the aircraft 10. The cabin is divided into zones, and at least one sensor is arranged in each zone. Sensors 22 are arranged in a lower part of the cabin (defined in relation to the ground), that is to say, at the floor of the cabin, for example beneath the seats, in an upper part of the cabin (defined in relation to the ground), that is to say, at the ceiling of the cabin, for example at the baggage compartments, and in an intermediate part located between the lower part and the upper part, that is to say, at the seats.

Each sensor 22 is configured to acquire, in real time, data 24 that are representative of the temperature of the cabin. A data item 24 that is representative of the temperature comprises, for example, the temperature itself, the flow rate of the air, the pressure in the cabin, etc. When a sensor 22 takes a measurement, the sensor 22 also stores the date and time of the measurement. In particular, each sensor 22 is configured to acquire data 24 that are representative of the temperature of the zone of the cabin in which the sensor 22 is arranged. Thus, the cabin of the aircraft is divided into zones, and sensors 22 make it possible to take the temperature of each of these zones. The sensors 22 are arranged in such a way that it is possible to take the temperature in the entire cabin. Thus, the sensors 22 register any differences in temperature between the various zones of the cabin. Indeed, and as is known, the temperature of the air in the upper part of the cabin is higher than in the lower part of the cabin.

Each sensor 22 comprises a transmitter (not shown in the figures) and is configured to send the acquired data 24 using its transmitter. The data from the sensors 22 are sent by wireless transmission.

The system 20 also comprises a computer server 26 (also known as a “cloud”). The computer server 26 comprises a receiver (not shown in the figures) and is configured to receive, in real time, the data 24 from the sensors 22, by means of its receiver.

The computer server 26 comprises a database 28 containing, for the aircraft 10, a predetermined cycle 40 of regulation of the temperature of the air of the cabin to a predetermined temperature during a given duration. This regulation cycle corresponds to a cycle of disinfecting the cabin of the aircraft.

Thus, for a given aircraft 10, the database 28 comprises the data 24 from the sensors 22 installed in the given aircraft 10, and the predetermined cycle 40 linked to the aircraft 10.

The system 20 also comprises a user interface 30 connected to the computer server 26 via a wireless connection. The user interface 30 is configured to automatically access the data 24 of the computer server 26, and to automatically display these to a user of the system 20.

The computer server 26 therefore comprises a transmitter (not shown in the figures) and is configured to send the data 24 from the sensors 22 to the user interface 30, by means of its transmitter. The user interface 30 therefore comprises a receiver (not shown in the figures) and is configured to receive the data 24 from the sensors 22 sent by the computer server 26, by means of its receiver.

The system 20 also comprises a ground pre-conditioned air unit 32, which is external to the aircraft 10.

The term “pre-conditioned air” is to be understood as air whose temperature is controlled in order to obtain the predetermined temperature.

The pre-conditioned air unit 32 comprises a pre-conditioned air generator 34 for the purpose of generating pre-conditioned air in the cabin, and a control module 36 connected the pre-conditioned air generator 34.

Thus, the user interface 30 comprises a transmitter (not shown in the figures) and is configured in such a way that, once the user interface 30 has access to the data 24 of the computer server 26, it transmits a control signal 38 to the control module 36.

The control module 36 comprises a receiver (not shown in the figures) and is configured in such a way that, upon receiving the control signal 38, it receives, in real time, the data from the computer server 26, by means of its receiver. The data from the computer server 26 include the data 24 from the sensors 22 and the predetermined cycle 40. The data are sent from the computer server 26 to the control module 36 by wireless transmission. The control module 36 is configured to control, in real time, the pre-conditioned air generator 34 according to the predetermined cycle 40.

Depending on the data 24, from the sensors 22, received by the control module 36, the predetermined cycle 40 is modulated in real time. The control module 36 thus controls, in real time, the pre-conditioned air generator 34 according to the modulated predetermined cycle. The pre-conditioned air generator 34 thus generates air in the cabin of the aircraft 10 with a predetermined temperature setpoint, a predetermined pre-conditioned air flow rate setpoint, and a predetermined duration setpoint, so as to conform to the modulated predetermined cycle.

The control module 36 comprises a transmitter (not shown in the figures) and is configured in such a way that it sends to the computer server 26, in real time and using its transmitter, information relating to the modulated predetermined cycle 62.

The control module 36 also sends, to the computer server 26, location data and an indication relating to the use of the pre-conditioned air unit 32.

Once the modulated predetermined cycle has been completed, the control module 36 stops controlling the pre-conditioned air generator 34, and the pre-conditioned air generator 34 stops. The control module 36 then sends, to the computer server 26, an indication relating to the end of the use of the pre-conditioned air unit 32.

At the end of the regulation cycle, the cabin of the aircraft 10 is thus disinfected, by the temperature being regulated to the predetermined temperature for the given duration.

Thus, this system 20 serves to generate air in the cabin of an aircraft, at a predetermined temperature and for a given duration, this being done automatically in order to disinfect the cabin of the aircraft.

Furthermore, since the sensors 22 are arranged in various zones of the cabin, and since the regulation cycle is modulated in real time by the data 24 from the sensors 22, the system 20 makes it possible to disinfect all the zones of the cabin, since the temperature of each zone of the cabin will have been regulated according to the predetermined cycle. Indeed, with the system 20, the temperature of each zone of the cabin will have been at the predetermined temperature for the given duration.

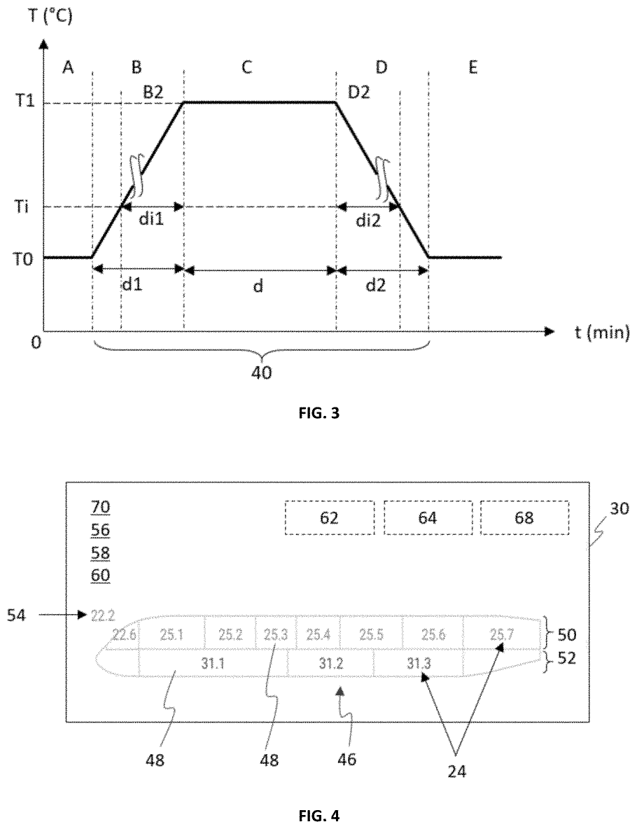

FIG. 3 illustrates the predetermined cycle 40 for regulating the temperature of the air of the cabin to a predetermined temperature for a given duration. The predetermined cycle 40 is defined by:

a first phase B of raising (or lowering) the temperature from an initial temperature T0, to a predetermined temperature T1, over a given duration d1;

a second phase C of holding the predetermined temperature T1 for a given duration d;

a third phase D of lowering (or raising) from the predetermined temperature T1 to a final temperature which corresponds to the initial temperature T0, over a given duration d2.

If the regulation cycle requires that the air of the cabin be heated to the predetermined temperature T1, which is above the initial temperature T0 of the cabin, the first phase B is an increase in temperature, and the third phase D is a reduction in temperature. Conversely, if the regulation cycle requires that the air of the cabin be cooled to the predetermined temperature T1, which is below the initial temperature T0 of the cabin, the first phase B is a reduction in temperature, and the third phase D is an increase in temperature.

Phase B corresponds to initiation of the disinfection of the cabin, phase C corresponds to the disinfection of the cabin, and phase D corresponds to initiation of the end of the disinfection of the cabin.

Prior to the predetermined cycle (phase A in FIG. 3), the temperature of the cabin is equal to the initial temperature T0, and after the predetermined cycle (phase E in FIG. 3), the temperature of the cabin is equal to the initial temperature T0.

The initial temperature of the cabin can be different from the initial temperature T0 defined according to the predetermined cycle. For example, the initial temperature of the cabin is equal to a temperature Ti, between the initial temperature T0 of the predetermined cycle, and the predetermined temperature T1. The information relating to the initial temperature Ti of the cabin is given by the data 24 from the sensors 22, which are received by the control module 36.

In this case, the predetermined cycle 40 is modulated.

The modulated predetermined cycle is then defined by a first phase B2 of raising (or lowering) the temperature, on the basis of the initial temperature Ti, until the predetermined temperature T1, over a given duration di1; then by the second phase C and the third phase D as defined hereinabove.

As a variant, the modulated predetermined cycle is defined by a first phase B2 of raising (or lowering) the temperature from the initial temperature Ti, until the predetermined temperature T1 is reached, over a given duration di1; then by the second phase C as defined hereinabove, and finally by a third phase D2 of lowering (or raising) from the predetermined temperature T1 to a final temperature which corresponds to the initial temperature Ti, over a given duration di2.

Thus, the control module 36, upon receiving the data 24 from the sensors 22 and the predetermined cycle 40, modulates the predetermined cycle 40, that is to say, determines the starting point and the end point of the predetermined cycle 40 to be considered in order to regulate the temperature of the cabin of the aircraft 10.

According to one configuration, the predetermined cycle 40 defines a predetermined temperature T1 between 56° C. and 68° C., and a given duration d between 15 minutes and 40 minutes. Thus, the predetermined cycle is a cycle of heating the cabin of the aircraft. Of course, a cycle of cooling (or air-conditioning) of the cabin of the aircraft can also be implemented.

According to one embodiment, shown in FIG. 2, the user interface 30 is an electronic device such as a mobile phone, a tablet or a computer. More specifically, the user interface 30 is an application on one of these electronic devices. Thus, the user interface 30 is arranged at a distance from the pre-conditioned air unit 32. Thus, the disinfection of a cabin of an aircraft is controlled automatically.

FIG. 4 shows an example of a user interface 30, which is configured so as to display a simplified model 46 of the cabin of the aircraft, which model shows the zones 48 of the cabin, in the upper part 50 and the lower part 52 of the cabin. The user interface 30 also displays, in real time, the data 24 from the sensors, the date 56 and the time 58 of the data 24, the external temperature 54 outside the aircraft, the location 60 (GPS data—short for Global Positioning System) of the aircraft, and the modulated predetermined cycle 62 received by the computer server 26. Thus, a user has access, in real time, to an indication of the phase of the regulation cycle that is currently underway.

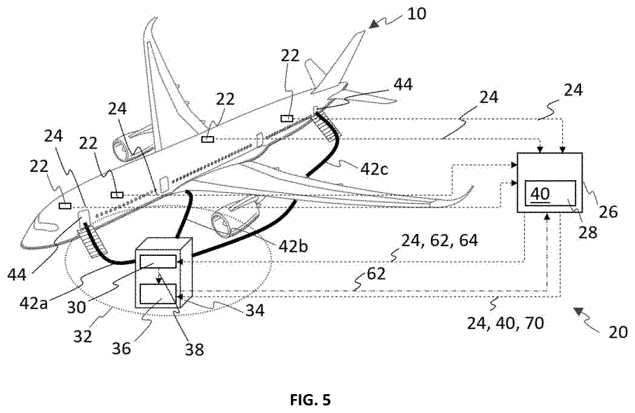

According to another embodiment, shown in FIG. 5, the pre-conditioned air unit 32 includes the user interface 30. The user interface 30 is then a human-machine interface that is directly integrated into the pre-conditioned air unit 32. Thus, the disinfection of a cabin of an aircraft is controlled manually.

According to a configuration shown in FIG. 2, the pre-conditioned air generator 34 comprises multiple flexible air lines 42a, 42b, 42c that are connected to the aircraft 10, and, in particular, to the access doors 44 of the aircraft 10. Of course, this configuration is non-limiting, and the pre-conditioned air generator 34 may comprise one, two or more than three flexible air lines connected to the aircraft 10.

According to one configuration, the pre-conditioned air unit 32 comprises a reservoir 32a containing at least one disinfectant solution. A disinfectant solution comprises at least one disinfectant product, that is to say, a product that can be used to disinfect a surface or the environment where the disinfectant solution is dispersed, that is to say, to deactivate the viruses present on that surface or in the environment where the disinfectant solution is dispersed. The reservoir is connected to an air outlet of the pre-conditioned air generator 34. The pre-conditioned air generator 34 is configured to generate a mixture comprising pre-conditioned air and one or more disinfectant products.

According to one embodiment, the control module 36 comprises first safety means 36a, which are configured to compare the value of each data item 24, from the sensors 22 and transmitted by the computer server 26, with a first predetermined threshold, and to stop controlling the pre-conditioned air generator 34 when the value of at least one data item 24 is above the first predetermined threshold. More specifically, the first safety means 36a comprise a comparison submodule 36b which is configured to compare the value of each data item 24, from the sensors 22 and transmitted by the computer server 26, with a first predetermined threshold, and a stopping submodule 36c that is configured to stop the operation of the pre-conditioned air generator 34 when the value of at least one data item 24 is above the first predetermined threshold. The comparison submodule 36b can take the form of a comparator, and the stopping submodule 36c can take the form of a switch or an actuator. The pre-conditioned air generator 34 then stops generating pre-conditioned air in the cabin. These first safety means 36a serve to protect the equipment in the cabin of the aircraft 10 from overheating. Indeed, certain cabin equipment, such as the air ducts, are designed to withstand a maximum temperature of approximately 70° C. The first safety means serve to avoid this maximum temperature being reached during the predetermined cycle.

According to another embodiment, the control module 36 comprises second safety means (not shown in the figures), which are configured to compare the value of each data item 24, from the sensors 22 and transmitted by the computer server 26, with a second predetermined threshold, and to stop the operation of the pre-conditioned air generator 34 when the value of at least one data item 24 is below the second predetermined threshold. More specifically, the second safety means comprise a comparison submodule (not shown in the figures), such as a comparator, which is configured to compare the value of each data item 24, from the sensors 22 and transmitted by the computer server 26, with a first predetermined threshold, and a stopping submodule (not shown in the figures), such as a switch or an actuator, that is configured to stop the operation of the pre-conditioned air generator 34 when the value of at least one data item 24 is above the first predetermined threshold. The pre-conditioned air generator 34 then stops generating pre-conditioned air in the cabin. These second safety means serve to protect the equipment in the cabin of the aircraft 10 from excessive cooling. Indeed, certain equipment in the cabin may be designed to withstand a minimum temperature that is below the second predetermined threshold. The second safety means make it possible to avoid this minimum temperature being reached during the predetermined cycle.

When a regulation cycle is interrupted upon activation of the safety means, an information item 64 relating to interrupting the predetermined cycle is transmitted, in real time, by the control module 36 to the computer server 26. This information item 64 is then displayed on the user interface 30. Thus, the user has access, in real time, to the information relating to interruption of the regulation cycle.

According to one embodiment, shown in FIG. 2, the system 20 comprises a safety device 66 which comprises sensors (not shown in the figures) deployed in multiple zones of the cabin and configured to acquire, in real time, data representative of the temperature of the various zones of the cabin, a comparator (not shown in the figures) configured to receive, in real time, the data acquired by these sensors and to compare, in real time, the value of these data to a predetermined threshold, and a transmitter (not shown in the figures) configured to send, in real time, a stop signal 68 when the value of a data item from one of these sensors is above the predetermined threshold. The stop signal 68 is sent in real time by the safety device 66 to the computer server 26, which sends the stop signal 68 in real time to the control module 36. Upon receiving the stop signal 68, the control module 36 stops controlling the pre-conditioned air generator 34, which then stops generating pre-conditioned air in the cabin. The stop signal 68 is also sent by the computer server 26 to the user interface 30, which displays this signal. The safety device 66 is mobile in the various zones of the cabin. This safety device 66 is independent of the safety means of the control module 36, is autonomous and permits redundancy of the safety means guarding against the risk of overheating of the cabin.

According to one embodiment, in order to connect to the user interface 30, a user provides a user identifier and a password which are unique to that user. Parameters for access to the data of the computer server 26 are associated with the user identifier. For example, a user may have access only to certain data in the database 28 of the computer server 26.

According to one embodiment, the aircraft is provided with an identifier, such as a QR (Quick Response) code or an NFC (Near-Field Communication) tag. The identifier may also be the number of the aircraft. The identifier of the aircraft provides unique identification of that aircraft. The identifier of the aircraft corresponds to information that provides unique identification of the aircraft among other aircraft.

The sensors 22 are configured to transmit the data 24 to the computer server 26 with an indication of the identifier of the aircraft. The computer server 26 receives the data 24 from the sensors and stores them in the database 28 with the indication of the identifier of the aircraft.

The user interface 30 comprises means 30a for detecting an identifier. These detection means may comprise a camera, or a sensor, which interacts with an identifier of an aircraft so as to identify one aircraft among a plurality of aircraft.

According to one configuration, detecting the identifier of the aircraft involves activating an NFC marker of a mobile phone on which the user interface 30 is installed, and receiving the NFC tag of the aircraft.

According to another configuration, detecting the identifier of the aircraft involves reading the QR code using a mobile phone on which the user interface 30 is installed.

With the identifier (QR code or NFC marker) of the aircraft, the user interface 30 receives a location datum of the aircraft, which is displayed (reference 60 in FIG. 4).

Once both the user and the aircraft have been identified, the user interface 30 is configured to automatically access the data of the computer server 26 only for the identified aircraft, and to display these automatically. The user interface 30 also displays the identifier 70 of the aircraft (as shown in FIG. 4). The identification of the aircraft serves to strengthen the security of the system 20, by allowing access to the data 24 from the sensors only once the user has been identified, and only for the identified aircraft.

Once both the user and the aircraft have been identified, the user interface 30 is configured to transmit the identifier 70 of the aircraft, together with the control signal 38, to the control module 36.

The control module 36 is configured to receive, in real time, only those data in the computer server 26 that are linked to the identifier 70 of the aircraft.

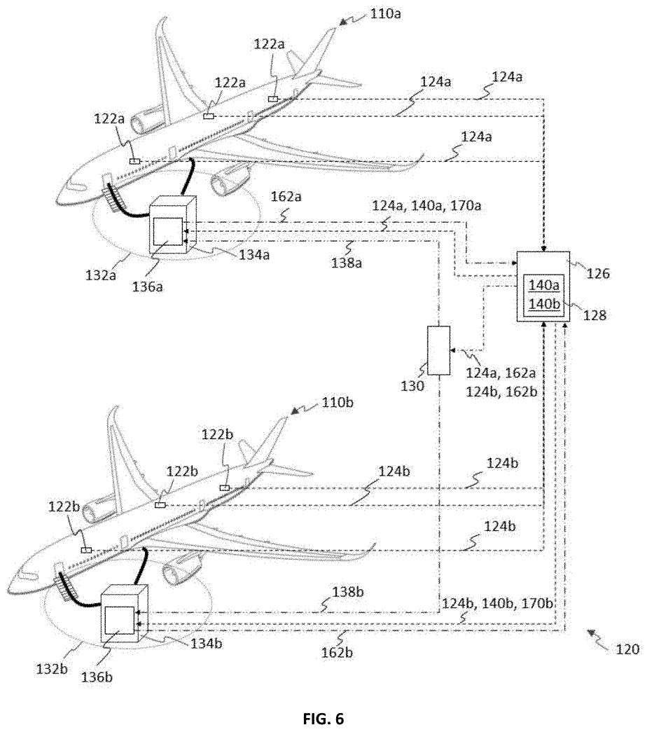

FIG. 6 shows a system 120 for regulating the temperature of the cabin of a fleet of aircraft 110a, 110b that are on the ground. A fleet of aircraft comprises a plurality of aircraft that are of the same type or of different types. The type of an aircraft is defined by the model of the aircraft, that is to say, the number of passengers that the aircraft can accommodate and the number of kilometers that the aircraft can travel without refueling. For example, in FIG. 6 the aircraft 110a is of a first type and the aircraft 110b is of a second type that is different from the first type.

Each aircraft 110a, 110b is provided with its own unique identifier 170a, 170b.

Each aircraft 110a, 110b comprises a plurality of sensors 122a, 122b installed in various zones of its cabin. The sensors 122a, 122b operate in the same way as the sensors 22 described previously.

A pre-conditioned air unit 132a, 132b, which is external to the aircraft 110a, 110b, is connected to each aircraft 110a, 110b. Each pre-conditioned air unit 132a, 132b comprises a pre-conditioned air generator 134a, 134b connected to a control module 136a, 136b. Each pre-conditioned air unit 132a, 132b operates in the same way as the pre-conditioned air unit 32 described previously.

The sensors 122a, 122b of each aircraft 110a, 110b are configured to acquire, in real time, and transmit data 124a, 124b to a computer server 126, with an indication of its identifier 170a, 170b. A single computer server 126 receives, in real time, the data 124a, 124b of the sensors 122a, 122b of the aircraft 110a, 110b.

The computer server 126 comprises a database 128 containing, for each aircraft 110a, 110b, a predetermined cycle 140a, 140b of regulation of the temperature of the air of the cabin during a given duration. The predetermined cycle 140a, 140b depends on the type of the aircraft 110a, 110b. Since the aircraft 110a, 110b are of different types, the predetermined cycle 140a for the aircraft 110a is different from the predetermined cycle 140b for the aircraft 110b.

Thus, for each aircraft 110a, 110b, the database 128 contains the data 124a, 124b from the sensors 122a, 122b and the predetermined cycle 140a, 140b.

The system 120 also comprises a user interface 130 connected to the computer server 126. The user interface 130 may be unique, or the system 120 may comprise multiple user interfaces 130. In FIG. 6, the user interface 130 is an application for a mobile phone, a tablet or a computer. A user connects to the user interface 130 using their user ID and their password.

Then, the aircraft 110a, 110b for which the user wishes to regulate the temperature of the cabin, for example the aircraft 110a, is identified. To that end, the identifier 170a of the aircraft 110a is detected, for example by reading the QR code of the aircraft 110a, or by activating the NFC marker of the aircraft 110a, using the detection means of the user interface 130.

Once the aircraft 110a has been identified, the user interface 130 is configured to access the data 124a from the sensors 122a of the aircraft 110a, which are stored on the computer server 126. The user interface 130 does not allow access to the other data in the database 128. The user is allowed access only to the data 124a linked to the identifier 170a of the aircraft 110a. The user interface 130 is configured to automatically display these data 124a along with the identifier 170a of the aircraft 110a.

The user, on the basis of the identifier 170a of the aircraft 110a, has access, via the user interface 130, to the location of the aircraft 110a. The user also has access, via the user interface 130 and the computer server 126, to the location data of the pre-conditioned air units of the airport where the aircraft 110a is parked, and to an indication relating to their current state of use. The location data of the pre-conditioned air units correspond to their last known location (location data sent to the computer server 126 during a previous use). The user can then select the pre-conditioned air unit, and thus the control module, to which the user interface 130 will send a control signal 138a, depending on the location data of the pre-conditioned air units and depending on their availability. For example, the user selects the pre-conditioned air unit 132a which is available, that is to say, which is not currently being used, and which is closest to the aircraft 110a.

In order to disinfect the cabin of the aircraft 110a, the user uses the user interface 130 to issue an instruction for a control signal 138a to be sent to the control module 136a.

Upon receiving the control signal 138a, the control module 136a receives, in real time, the data from the computer server 126. The data from the computer server 126 include the data 124a from the sensors 122a and the predetermined cycle 140a. The control module 136a controls, in real time, the pre-conditioned air generator 134a according to the predetermined cycle 140a, or according to the modulated predetermined cycle 162a on the basis of the data 124a. The pre-conditioned air generator 134a thus generates air in the cabin of the aircraft 110a with a predetermined temperature setpoint, a predetermined pre-conditioned air flow rate setpoint, and a predetermined duration setpoint, so as to conform to the modulated predetermined cycle 162a.

Once the control signal 138a has been sent to the control module 136a, the user, using the user interface 130, identifies the aircraft 110b whose cabin temperature the user wishes to regulate. The identification of the second aircraft 110b does not interrupt the predetermined cycle that is underway for the first aircraft 110a.

Once the aircraft 110b has been identified, the user interface 130 is configured to access the data 124b from the sensors 122b of the aircraft 110b, which are stored on the computer server 126. The user interface 130 automatically displays these data 124b along with the identifier 170b of the aircraft 110b.

The user, on the basis of the identifier 170b of the aircraft 110b, has access, via the user interface 130, to the location of the aircraft 110b, and to the location data of the pre-conditioned air units of the airport where the aircraft 110b is parked, and to an indication relating to their current state of use. The user can then select the pre-conditioned air unit to which the user interface 130 will send a control signal 138b, via its control module, depending on the location data of the pre-conditioned air units and depending on their availability. For example, the user selects the pre-conditioned air unit 132b which is available and which is closest to the aircraft 110b.

In order to disinfect the cabin of the aircraft 110b, the user uses the user interface 130 to issue an instruction for a control signal 138b to be sent to the module 136b.

Upon receiving the control signal 138b, the control module 136b receives, in real time, the data from the computer server 126. The control module 136b controls, in real time, the pre-conditioned air generator 134b according to the predetermined cycle 140b, or according to the modulated predetermined cycle 162b on the basis of the data 124b. The pre-conditioned air generator 134b thus generates air in the cabin of the aircraft 110b with a predetermined temperature setpoint, a predetermined pre-conditioned air flow rate setpoint, and a predetermined duration setpoint, so as to conform to the modulated predetermined cycle 162b.

For each aircraft 110a, 110b, once the predetermined cycle has been completed, the control module 136a, 136b stops controlling the pre-conditioned air generator 134a, 134b, and the pre-conditioned air generator 134a, 134b stops. The cabin of each aircraft 110a, 110b is then disinfected.

At any point, the user may, via the user interface 130, stop the disinfection of an aircraft, by sending a stop signal to the control module 136a, 136b.

A user may thus, using a single user interface 130, simultaneously regulate, in real time and automatically, the temperature of the cabin of multiple aircraft 110a, 110b of a fleet.

In particular, the user uses a computer program product comprising a set of program code instructions that, when these instructions are executed by a processor 30b, configure the processor to implement a method for regulating the temperature of the cabin of an aircraft that is on the ground, as described above.

The invention has been described for regulation (heating or cooling) of the temperature of the cabin of an aircraft, using ground support equipment (or GSE) in the form of a pre-conditioned air unit. Of course, the principle of the invention can also be implemented using any other ground support equipment, such as the water supply and disposal unit, or the refueling unit.

The systems and devices described herein may include a controller or a computing device comprising a processing unit and a memory which has stored therein computer-executable instructions for implementing the processes described herein. The processing unit may comprise any suitable devices configured to cause a series of steps to be performed so as to implement the method such that instructions, when executed by the computing device or other programmable apparatus, may cause the functions/acts/steps specified in the methods described herein to be executed. The processing unit may comprise, for example, any type of general-purpose microprocessor or microcontroller, a digital signal processing (DSP) processor, a central processing unit (CPU), an integrated circuit, a field programmable gate array (FPGA), a reconfigurable processor, other suitably programmed or programmable logic circuits, or any combination thereof.

The memory may be any suitable known or other machine-readable storage medium. The memory may comprise non-transitory computer readable storage medium such as, for example, but not limited to, an electronic, magnetic, optical, electromagnetic, infrared, or semiconductor system, apparatus, or device, or any suitable combination of the foregoing. The memory may include a suitable combination of any type of computer memory that is located either internally or externally to the device such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. The memory may comprise any storage means (e.g., devices) suitable for retrievably storing the computer-executable instructions executable by processing unit.

The methods and systems described herein may be implemented in a high-level procedural or object-oriented programming or scripting language, or a combination thereof, to communicate with or assist in the operation of the controller or computing device. Alternatively, the methods and systems described herein may be implemented in assembly or machine language. The language may be a compiled or interpreted language. Program code for implementing the methods and systems for detecting skew in a wing slat of an aircraft described herein may be stored on the storage media or the device, for example a ROM, a magnetic disk, an optical disc, a flash drive, or any other suitable storage media or device. The program code may be readable by a general or special-purpose programmable computer for configuring and operating the computer when the storage media or device is read by the computer to perform the procedures described herein.

Computer-executable instructions may be in many forms, including program modules, executed by one or more computers or other devices. Generally, program modules include routines, programs, objects, components, data structures, etc., that perform particular tasks or implement particular abstract data types. Typically, the functionality of the program modules may be combined or distributed as desired in various embodiments.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

1. A system for regulating the temperature of a cabin of an aircraft when on the ground, comprising:
a plurality of sensors arranged in various zones of the cabin, each sensor being configured to acquire, in real time, data representative of the temperature of the zone of the cabin,
a computer server configured to receive, in real time, the data from the plurality of sensors and comprising a database containing, for the aircraft, a predetermined cycle of regulation of the temperature of the air of the cabin to a predetermined temperature during a given duration,
a ground pre-conditioned air unit comprising:
a pre-conditioned air generator for generating pre-conditioned air in the cabin, and
a control module connected to the pre-conditioned air generator and configured to receive the data from the computer server,
a user interface connected to the computer server, and configured to access the data from the computer server and to transmit a control signal to the control module,
the control module being configured such that, upon receiving the control signal, it controls, in real time, the pre-conditioned air generator, according to the predetermined cycle, the predetermined cycle being modulated, in real time, depending on the data from the computer server.

2. The system according to claim 1, wherein the user interface is a portable telephone, a tablet or a computer.

3. The system according to claim 1, wherein the pre-conditioned air unit includes the user interface.

4. The system according to claim 1, wherein the control module is configured such that, upon receiving the control signal, the control module sends the data relating to a location of the control module to the computer server.

5. The system according to claim 1, wherein the pre-conditioned air unit comprises a reservoir containing at least one disinfectant solution connected to the pre-conditioned air generator, and the pre-conditioned air generator is configured to generate a mixture comprising pre-conditioned air and the disinfectant solution.

6. The system according to claim 1, wherein the control module comprises safety means comprising a comparison submodule that is configured to compare a value of each data item from the computer server with a predetermined threshold, and a stopping submodule that is configured to stop control of the pre-conditioned air generator when the value of at least one data item from the computer server is above the predetermined threshold.

7. The system according to claim 1, wherein the aircraft is fitted with an identifier, and wherein the user interface comprises means for detecting the identifier, and is configured such that, upon detecting the identifier of the aircraft, the user interface accesses the data of the computer server for the identified aircraft.

8. A method for regulating a temperature of a cabin of an aircraft when on the ground, using a regulation system comprising a plurality of sensors arranged in various zones of the cabin, a computer server comprising a database containing, for the aircraft, a predetermined cycle of regulation of the temperature of the air of the cabin to a predetermined temperature during a given duration, a user interface connected to the computer server, and a ground pre-conditioned air unit, which comprises a pre-conditioned air generator and a control module connected to the pre-conditioned air generator, the method comprising the following steps:
acquiring, in real time and by means of the plurality of sensors, data representative of the temperature of various zones of the cabin,
receiving, in real time, the data from the plurality of sensors by means of the computer server,
accessing, via the user interface, the data from the computer server,
transmitting, via the user interface, a control signal, to the control module, and
upon receiving the control signal:
receiving, by the control module and in real time, the data from the computer server,
modulating, in real time, the predetermined cycle depending on the data from the computer server,
controlling, in real time, the pre-conditioned air generator, according to the modulated predetermined cycle.

9. The method according to claim 8, the aircraft being provided with an identifier, and the user interface comprising means for detecting an identifier, wherein the method comprises, prior to the step of accessing the data of the computer server, the steps of:
using the detection means to identify the aircraft by means of its identifier,
transmitting, via the user interface, the identifier of the aircraft to the control module.

10. A computer program product comprising a set of program code instructions that, when the instructions are executed by a processor, configure the processor to implement a method for regulating the temperature of the cabin of an aircraft according to claim 8.
